(19) United States
(12) Patent Application Publication
(10) Pub. No.: US 2020/0390422 A1
(43) Pub. Date: Dec. 17, 2020

(54) Toilet for Analysis of Settled Urine

(71) Applicant: Medic, Inc., Provo, UT (US)

(72) Inventors: David R. Hall, Provo, UT (US); Jared Reynolds, Spanish Fork, UT (US); Travis Niederhauser, Mapleton, UT (US); David Crismon, Herriman, UT (US); Joshua Larsen, Spanish Fork, UT (US)

(73) Assignee: Medic, Inc., Provo, UT (US)

(21) Appl. No.: 16/883,982

(22) Filed: May 26, 2020

Related U.S. Application Data
(60) Provisional application No. 62/862,569, filed on Jun. 17, 2019.
(60) Provisional application No. 62/888,690, filed on Aug. 19, 2019.
(60) Provisional application No. 62/888,704, filed on Aug. 19, 2019.

(51) Int. Cl. A61B 10/00; A61B 5/20; G01N 35/10; G01N 33/493; E03D 11/13; G01N 1/20

(52) U.S. Cl. CPC A61B 10/007; A61B 5/208; G01N 35/1002; A61B 2010/0009; E03D 11/13; G01N 1/20; G01N 33/493

(57) ABSTRACT

An analytical toilet comprising a bowl for receiving excreta from a user, a urine collection chamber in fluid communication with the bowl, a sensor for detecting properties of the urine after at least partial settling of the urine, a valve adapted to release the captured urine from the urine collection chamber, and a source of flush water to clean the urine collection chamber.

### CROSS-REFERENCE TO RELATED APPLICATIONS

This application claims the benefit under 35 U.S.C. 119(e) of U.S. Provisional Patent Application Nos. 62/862,569 filed Jun. 17, 2019, 62/888,690 filed Aug. 19, 2019 and 62/888,704 filed Aug. 19, 2019. The disclosures of each of said applications are hereby incorporated herein by reference.

### TECHNICAL FIELD

The present disclosure relates to analytical toilets. More particularly, it relates to analytical toilets equipped to provide health and wellness information related to excreta deposited by a user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Some medical information that can be detected from urine is overlooked because urine samples are infrequently taken, and many analyses are performed on urine samples that are in various stages of settling which adds variability from test to test. Ideally, the optimal times to analyze a sample of urine is immediately after excretion by a user or after a pre-determined settling time. Allowing urine samples to settle for uniform and pre-determined times and to observe the settling process over the pre-determined time aids in better understanding the health status and health trends of a user.

### SUMMARY

One aspect of the present invention, is an analytical toilet comprising a bowl for receiving excreta from a user, a urine collection chamber in fluid communication with the bowl, a sensor for detecting properties of the urine after at least partial settling of the urine, a valve adapted to release the captured urine from the urine collection chamber, and a source of flush water to clean the urine collection chamber.

In another aspect, at least a portion of the urine collection chamber is transparent.

In still another aspect, the sensor further comprises one or more sensors along the height of the urine collection chamber are adapted to detect properties of different sections of the at least partially settled urine.

In a still further aspect, the one or more sensors measures protein, viral, bacterial, illicit drug, pharmaceutical drug, blood, mucous, fat, chyle, leucocyte, epithelial cell, or sugar content in the urine.

In a yet still further aspect, the one or more sensors measures the specific gravity, resistivity, pH, density, salinity, or osmolality at different sections of the at least partially settled urine within the urine collection chamber.

In still yet another aspect of the invention, the one or more sensors spectroscopically measures the properties of different sections of the at least partially settled urine.

In another aspect, the analytical toilet includes an image capturing device and is positioned to capture images through the transparent portion of the urine collection chamber. The image capturing device can record the settling of the urine sample in the urine collection chamber over a pre-determined time period. The image capturing device can be configured to capture images along the vertical axis of the at least partially settled urine.

In still another aspect, further additional image capturing devices can be included, where the image capturing device and the additional image capturing devices are positioned at different heights along the urine collection chamber to record different sections of the at least partially settled urine.

In a still further aspect, the urine collection chamber further comprises a fluid level sensor device. The fluid level sensor can be used to measure the volume of urine added to the urine collection chamber and the rate at which urine is added to the sample collection chamber during a urination event by a user to thereby determine a urination profile.

In a yet still further aspect, the analytical toilet further includes an ion detection device, variable light path spectroscopic analysis system, electrodes, or a drawing port.

In still yet another aspect of the invention, the analytical toilet further includes a filter to prevent feces from entering the urine collection chamber.

In another aspect of the present invention is, the analytical toilet further includes an overflow path for urine exceeding the volume of the urine collection chamber.

In another aspect, the analytical toilet further includes a heating element to drive off water and concentrate the urine sample. The heating element can be a resistive coil, IR heater, or a hot stage.

In still another aspect, the analytical toilet further includes a reagent dispensing device to dispense one or more reagents into the urine collection chamber. The reagent dispensing device can be a microfluidic, capillary, diaphragm, piston, screw, rotary, or peristaltic dispensing device.

In still yet another aspect, the one or more reagents dispensed by a reagent dispensing device includes a buffer solution, sulfosalicylic acid, CuSO₄, Benedict's reagent, sodium nitroprusside, or acetic acid.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1A illustrates an analytical toilet with the lid closed, according to an embodiment of the disclosure.

FIG. 1B illustrates an analytical toilet with lid open, according to an embodiment of the disclosure.

FIG. 1C illustrates an overhead view of an analytical toilet with lid open, according to an embodiment of the disclosure.

FIG. 2 illustrates a cross-sectional view of a urine analysis system in an analytical toilet, according to an embodiment of the disclosure.

FIG. 3 illustrates a cross-sectional view of a urine analysis system in an analytical toilet, according to an embodiment of the disclosure.

FIG. 4 illustrates a cross-sectional view of a urinalysis system in an analytical toilet, according to an embodiment of the disclosure.

FIG. 5 illustrates a cross-sectional view of a urine analysis system with a heating element in an analytical toilet, according to an embodiment of the disclosure.

FIG. 6 illustrates a cross-sectional view of a urinalysis system in an analytical toilet, according to an embodiment of the disclosure.

### DETAILED DESCRIPTION

### Overview

Embodiments of methods, materials and processes described herein are directed towards analytical toilets. Analytical toilets are equipped to provide health and wellness information related to excreta deposited by a user.

Analytical toilets can be used to analyze urine excreted by a user. The disclosure herein describes a system with various components to analyze a sample of urine.

### Definitions

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals. This includes a conventional toilet upon which a user sits (“western style”), as well as toilet over which a user squats (“eastern style”) and before which a user stands (“urinal”).

As used herein, the term “bowl” refers to the portion of a toilet that is designed to receive excreta.

As used herein, the term “user” refers to any individual who interacts with the toilet and deposits excreta therein.

As used herein, the term “excreta” refers to any substance released from the body including urine, feces, menstrual discharge, and anything contained or excreted therewith.

As used herein, the term “excretion profile” is meant to refer collectively to the rate of excretion at any moment in time of an excretion event and the total volume or mass of excreta as a function of time during an excretion event. The terms “defecation profile” and “urination profile” refer more specifically to the separate measurement of excreta from the anus and urethra, respectively.

As used herein, the term “manifold” is intended to have a relatively broad meaning, referring to a device with multiple conduits and valves to controllably distribute fluids, namely water, liquid sample and air.

As used herein, the term “settled” is intended to have a relatively broad meaning, referring to the condition where at least a significant portion of the particles in the urine have fallen to the bottom region of the captured urine sample.

As used herein, the term “solenoid valve” refers to an electrically activated valve, typically used to control the flow or direction of air or liquid in fluid power systems. Solenoid valves are the most frequently used control elements in fluidics. Solenoid valves are used to shut off, release, dose, distribute or mix fluids.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, spectrophotometer, colorimeter, refractometer, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, turbidimeters, flow cytometers, and analyzers.

As used herein, the term “imaging sensor” is meant to refer to any device for detecting and/or measuring a property of a person or of a substance that relies on electromagnetic radiation of any wavelength (e.g., visible light, infrared light, ultra-violet, x-ray) or sound waves (e.g., ultrasound) to view the surface or interior of a user or substance. The term “imaging sensor” does not require that an image or picture is created or stored even if the sensor is capable of creating an image.

As used herein, the term “image capturing device” is meant to refer to any device for capturing video images or still images. The captured images may be recorded and stored for later analysis. In some instances, the images may be viewed in real-time by a user or a health care professional. A video recording camera or a still picture camera are types of image capturing devices. The image capturing device may also illuminate the sample with a light or other means to better view the sample.

### Exemplary Embodiments

This invention allows for urine samples to be taken automatically to better detect different densities of particles in the urine and to trend this information over time. The urine sample can remain undisturbed for a pre-determined amount of time to allow the sample to settle, such as by gravity (e.g., gravimetrically). Urine samples may be analyzed quickly after a urination event by a user. Urine samples may be analyzed after only partial settling after short periods of time or after longer periods of time wherein the urine sample is substantially completely settled. The sample can be allowed to drain intermittently or slowly to allow scans of the urine at the different densities.

This urine capturing device would primarily be used in an analytical toilet where the entire urine sample could be collected without human interaction. The sampling area would clean, collect, analyze, and dispose of the urine sample without human interaction and without the need for the donator to use a toilet differently from normal.

Sensor scans of the urine can be taken of various density sections or regions of the urine by being placed at multiple levels along the vertical axis on the side of a urine sample collection chamber. The sensors can be placed at the base of the sample chamber and the sample can be drained intermittently or slowly to allow the sensors to take multiple readings of the various densities.

A urine sample may also be heated, or reagents added to the urine to collect other information.

A urine sample may be analyzed spectroscopically, chromatographically, or with an ion-selective electrode. Analytes such as ions, viruses, bacteria, proteins, blood, mucous, chyle, fat, epithelial cells, or sugars may be detected.

After desired readings are taken the urine would be allowed to drain into a sewer and the urine collection area would be rinsed and/or sanitized with cleansing fluids.

### Collection and Analysis of Urine

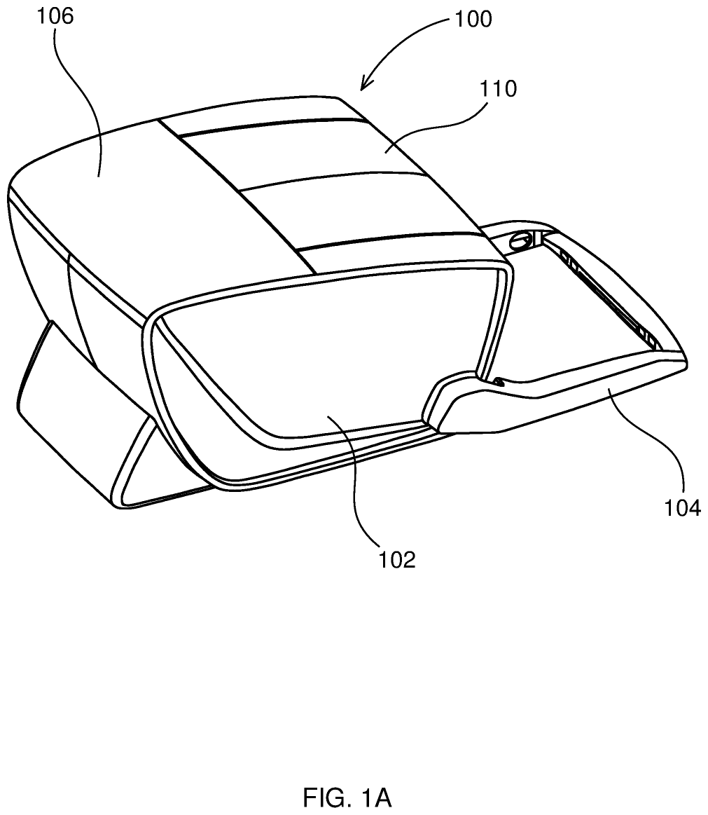

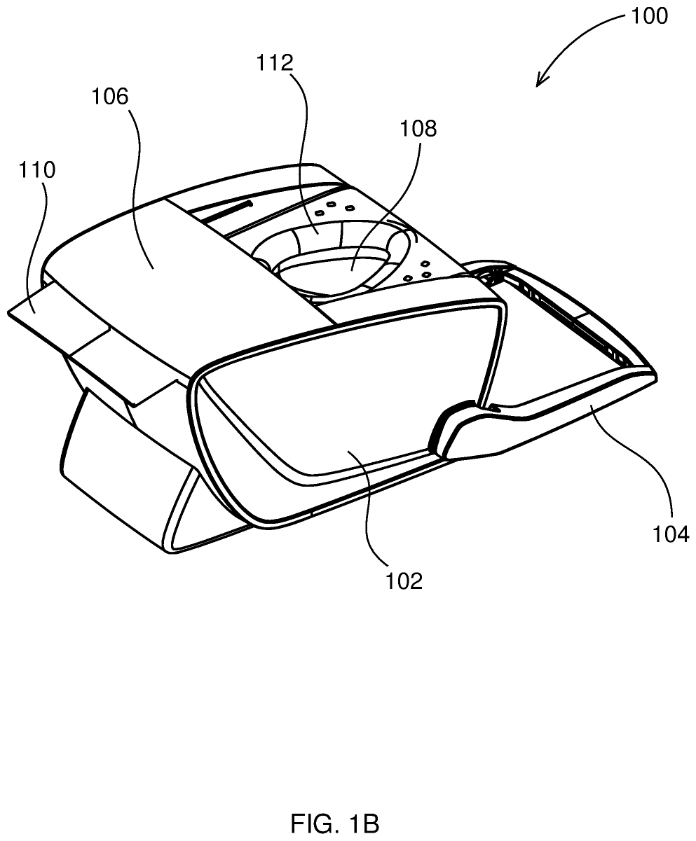

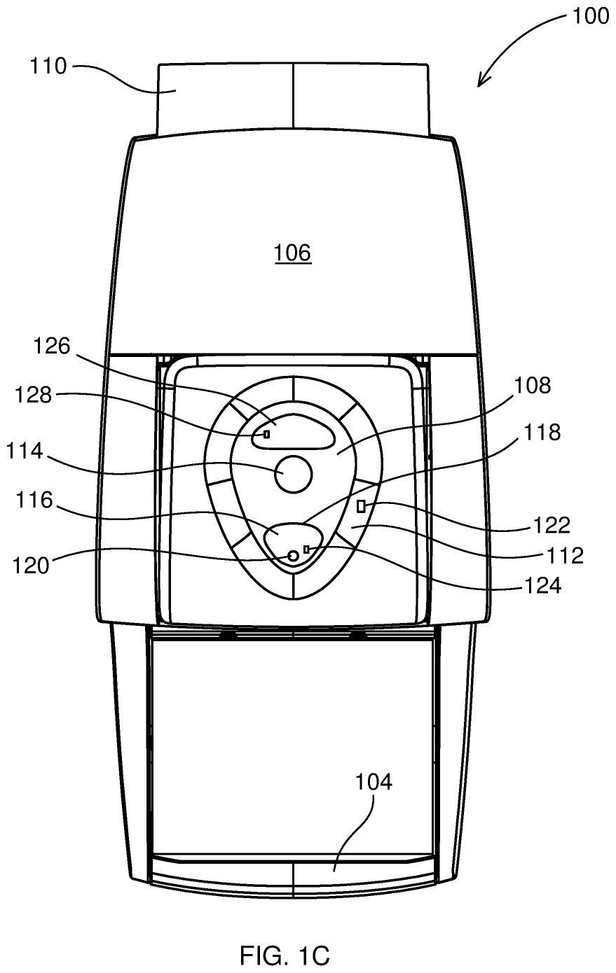

Now referring to FIGS. 1A-C, a preferred embodiment of an analytical toilet 100 is shown. FIG. 1A illustrates the analytical toilet 100 with the lid 110 closed, according to an embodiment of the disclosure. FIG. 1A further shows exterior shell 102, foot platform 104 and rear cover 106. The lid 110 is closed to prevent a user from depositing excreta in toilet 100 until the toilet is ready for use.

FIG. 1B illustrates toilet 100 with lid 110 open, according to an embodiment of the disclosure. Toilet 100 includes exterior shell 102, rear cover 106, bowl 108, seat 112, lid 110, and foot platform 104. Housed within toilet 100 are a variety of features, including equipment, that facilitate receiving excreta, processing urine for analysis, analyzing urine, and disposing of urine and any feces. FIG. 1B shows toilet 100 with lid 110 open so a user can sit on seat 112 and deposit excreta in toilet 100. Bowl 108 may be transparent, translucent, or opaque.

FIG. 1C illustrates an overhead view of an analytical toilet 100 with lid 110 open, according to an embodiment of the disclosure. Toilet 100 further includes a drain 114 where feces or urine may be removed and directed to a sewer or other location. Toilet 100 further includes a urine collection area 116 that is separated from the rest of bowl 108 by a partition 118. Urine collection 116 area further comprises a urine collection port 120. Port 120 may be a hole or a slit or other shape. Port 120 may comprise a filter or screen to prevent solid material, such as feces or toilet paper, from entering the port 120. The seat 112 may comprise one or more sensors 122. In some embodiments, there may be no sensors in the seat 112. The one or more sensors 122 may be located anywhere on the seat 112. Sensors 122 may be configured to measure body weight, body temperature, skin moisture, pulse, or other information about a user. The urine collection area 116 further comprise one or more sensors 124. Sensors 124 in urine collection area 116 may be used to determine temperature, detect solids, or other information about the urine from a user. In some instances, urine collection area 116 may not have one or more sensors.

Toilet 100 comprises a fecal shelf 126. Fecal shelf 126 is an area where feces can be received from a user, collected and analyzed. Fecal shelf 126 comprises one or more sensors, such as imaging sensors 128. The one or more imaging sensors 128 may be used to determine the shape or the form of the feces, detect parasites, blood, or other information.

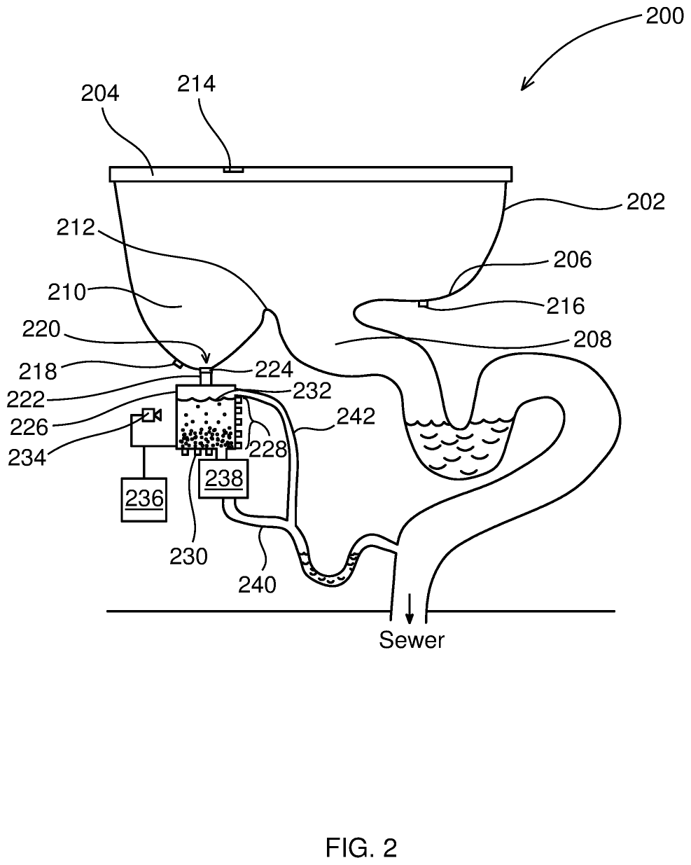

FIG. 2 illustrates a cross-sectional view of a urine analysis system in an analytical toilet 200, according to an embodiment of the disclosure. Analytical toilet 200 comprises a bowl 202, seat 204, fecal shelf 206, drain 208, urine collection area 210, and partition 212. Bowl 202 may be comprised of a ceramic such as porcelain, a metal, or a polymer. Drain 208 leads to and is in fluid communication with a sewer. A trap is preferably located between drain 208 and the sewer. Toilet 200 comprises one or more seat sensors 214, fecal shelf sensors 216, or urine collection area sensors 218. In some instances, there may be no seat sensors 214, fecal shelf sensors 216, or urine collection area sensors 218 in analytical toilet 200.

Toilet 200 illustrated in FIG. 2 further comprises a urine collection port 220 in fluid communication with a first fluid passage 222. Urine collection port 220, preferably located at the base of urine collection area 210, further comprises a device 224, such as a filter or mesh screen, to prevent solid material, such as feces or toilet paper, from passing through port 220. An infra-red (IR) sensor may be adjacent to collection port 220 or passage 222 to determine the temperature of the urine sample to understand core body temperature of the user. Toilet 200 comprises a urine sample collection chamber 226 (it should be known that throughout this disclosure, “urine sample collection chamber” may also be referred to as a “sample chamber” or a “urine collection chamber”). Sample chamber 226 is in fluid communication with urine collection area 210 and receives urine from a user that passes through port 220 and passage 222. Sample chamber 226 may be transparent or have a section that is transparent such as a window running along the vertical axis of the sample chamber. A transparent window may be located along a horizontal axis of sample chamber 226 on top, bottom, or both top and bottom of the chamber. Sample chamber 226 may be made of at least a portion of glass or a transparent polymer such as polycarbonate. Sample chamber 226 may be graduated to determine the volume of the urine sample deposited by a user. Sample chamber 226 may also be referred to as a sample chamber, receptacle, container, cylinder, or other receiving device.

Sample chamber 226 may comprise one or more sensors located along the vertical axis of the chamber. A plurality of sensors 228 arranged along the vertical axis at various or equidistant heights adjacent to the chamber 226 in toilet 200 illustrated in FIG. 2. Sample chamber 226 further comprises one or more sensors 230 arranged along the horizontal axis direction at various or equidistant locations along the bottom of the chamber 226. Horizontal axis sensors 230 may also be located on top of chamber 226. Sensor scans of the urine sample can be taken quickly after a urination event by a user. Sensor scans of the urine sample can be taken of various density sections or regions of the urine sample 232 as the urine sample 232 is allowed to settle over a period of time by being placed at multiple height locations on the side of the sample chamber 226 to determine properties of the settled urine at different sections. The urine can settle gravimetrically (i.e., by gravity) or by other means such as centrifugation. The settling time can vary such as from one second up to about 60 min or more. The settling time may be in the range of about 1-60 min. Preferably, the settling time may be in the range of about 5-30 min.

The sensors 230 can be placed at the base of the sample chamber 226 and the urine sample 232 can be drained intermittently or slowly to allow the sensors 228, 230 to take multiple readings of the various densities. The sensors 228, 230 may also measure temperature, salinity, pH, and osmolality of the urine sample at one or more height locations in the settled urine located in the sample chamber 226. Resistivity probes may be installed in order to measure the resistivity at various depths or sections of the urine sample along the vertical axis of the sample chamber 226. A spectrometer or spectrophotometer may be integrated with the sample chamber 226 to spectroscopically measure analytes in the urine sample. Sample chamber 226 may further comprise a thermometer or thermocouple to determine the temperature of the urine sample. The sensors 228, 230 may comprise one or more imaging sensors. In some embodiments, settling may be caused by a centrifuge to result in centrifugal settling.

In one embodiment, at least one moveable sensor may be located proximal to the sample chamber 226. The moveable sensor may move in an up and down and continuous manner along the vertical axis of the sample chamber 226. This may collect gradient data of the settled of the sample from top to bottom or bottom to top. The moveable sensor may be located on a track. This may be an alternative sensor design to having one or more stationary sensors placed in various locations along the vertical axis of the sample chamber 226 as previously described. The speed at which the moveable sensor moves along the vertical axis may be controlled automatically. Images of a urine sample may be taken continuously or intermittently through a window in the sample chamber 226. In another embodiment, a moveable sensor may move in a direction along the horizontal axis of the sample chamber 226.

Sample chamber 226 may further comprise fluid level sensors. The fluid level sensor may be used to determine volume a urine sample by measuring the height of the urine sample collected in the chamber 226. The fluid level sensors may comprise a magnetic float, mechanical float, proximity sensor, weight sensor, pressure sensor, pneumatic level sensor, conductive level sensor, or a microprocessor-controlled frequency state fluid level sensor. The fluid level sensors may be in electrical communication with a processor. A float sensor may be comprised of a polymeric material that has a density lower than water such that it can float on top of the urine sample 232. The float can be used to determine the flow rate at which the sample chamber fills up with urine and a final volume. The float may be equipped with a temperature sensing device such as a thermometer or thermocouple. A magnetic actuated float sensor may comprise a permanent magnet sealed inside a float wherein the float rises or falls to the fluid level. A mechanical actuated float senses a fluid level by movement against a miniature (micro) switch.

Analytical toilet 200 further comprises an image capturing device 234 in close proximity to the sample chamber 226. Image capturing device 234 may further comprise a light source to illuminate the urine sample 232. The light source may be able to illuminate the urine sample 232 at different frequencies. Image capturing device 234 may take still images or video. For example, image capturing device 234 may take video of a urine sample 232 as the sample is allowed to settle over a period of time. The images captured by the image capturing device 234 can be captured at various frequencies of lighting to emphasize different particles located in the urine sample 232. The visual images may be captured over time to detect the rate of separation of the particles in the urine sample 232. Toilet 200 further comprises a control system 236 to provide power to sensors 228, 230, image capturing device 234, or other components. System 236 may also be capable of providing processing and storage of the information collected by the sensors.

Sample chamber 226 is in fluid communication with a valve 238. Valve 238 allows for draining of the sample chamber 226 through a second fluid passage 240 and towards the sewer to dispose of the urine sample. In a preferred embodiment, valve 238 is a solenoid valve. Valve 238 can be used to drain the urine sample 232 quickly or may be controlled to drain the urine sample 232 at a carefully controlled rate. Other valves that may be used are ball valves, needle valves, butterfly valves, pinch valves, diaphragm valves, globe valves, angle body valves, or angle seat piston valves.

Second fluid passage 240 may include a trap before the passage connects into a sewer line. Analytical toilet 200 further comprises an overflow 242. Overflow 242 is used for a high-volume urine sample wherein the excess urine may be allowed to drain into passage 240 and into the sewer.

Analysis of a urine sample may be carried out using analytical toilet 200 as follows. A user urinates into the urine collection area 210. The urine collection area funnels the urine sample 232 toward urine collection port 220. The urine sample 232 passes through screen 224, into fluid passage 222 and collects in sample chamber 226. As the urine sample collects in the chamber 226, the sensors 228, 230 detect the rate at which the chamber is filled by the sample and takes a final volume reading once the fill rate reaches zero. Once the filling of the sample chamber 226 is completed, the urine is allowed to settle over a period of time. The settling time may be in the range of about 0.5-60 min. Over a shorter period of time, such as less than 10 min, the urine sample may only be partially settled. Over longer periods of time, such as 45-60 min, the sample may be substantially completely settled. In some instances, the settling time may be longer. Only partial settling may be necessary depending on the testing required. The sensors continue to monitor the urine while the image capturing device 234 records the settling process. The image capturing device 234 may also illuminate the urine sample for better viewing and clarity of the images taken of the settling process. The sensors may collect data on a sample of urine that has only partially settled over a shorter period of time. In other instances, the sensors may collect data on a sample of urine that has substantially completely settled over a longer period of time. Once the pre-determined settling time is completed and the sensors collect the information, the valve 238 opens and allows the urine sample 232 to pass through passage 240 and be drained into the sewer. The urine sample can be drained intermittently or slowly to allow the sensors at the base of the sample chamber to take multiple readings of the various densities. Once the urine sample is drained, the urine collection area 210, passage 222, filter 224, sample chamber 226, and valve 238 is rinsed and/or sanitized with cleansing fluids.

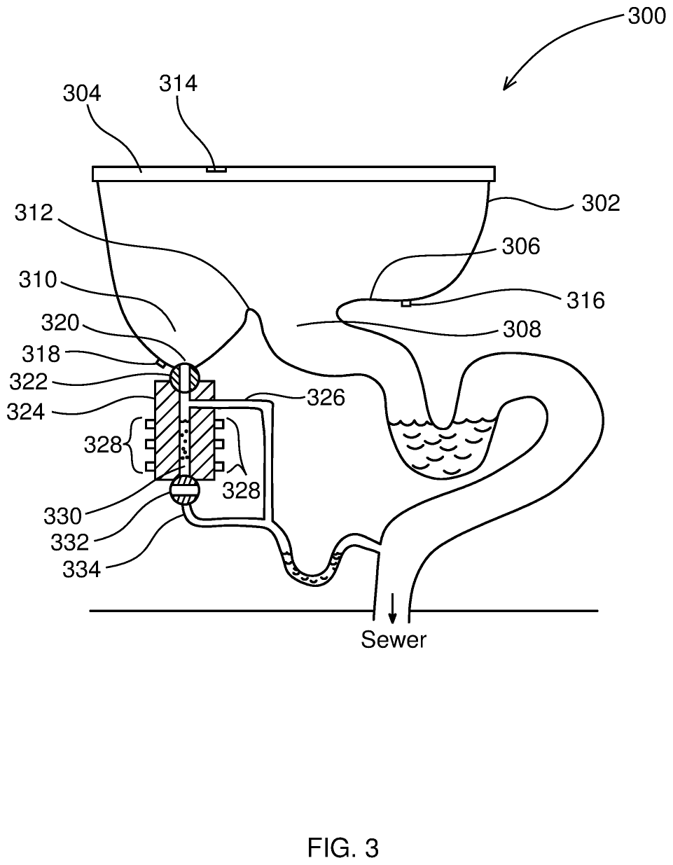

FIG. 3 illustrates a cross-sectional view of a urine analysis system in an analytical toilet 300, according to an embodiment of the disclosure. Analytical toilet 300 comprises a bowl 302, seat 304, fecal shelf 306, drain 308, urine collection area 310, partition 312, one or more seat sensors 314, fecal shelf sensors 316, or urine collection area sensors 318. In some instances, there may be no seat sensors 314, fecal shelf sensors 316, or urine collection area sensors 318 in analytical toilet 300.

Toilet 300 illustrated in FIG. 3 further comprises a urine collection port 320 that is preferably located at the base of urine collection area 310. Port 320 may further comprise a device such as a filter or mesh screen to prevent solid material, such as feces or toilet paper, from passing through port 320. An infra-red (IR) sensor may be adjacent to collection port 320 to determine the temperature of the urine sample to understand core body temperature of the user. Toilet 300 comprises a first valve 322 that is in fluid communication with bowl 302, area 310, and port 320. In a preferred embodiment, valve 322 is a ball valve as depicted in FIG. 3. Valve 322 is in fluid communication with and adjacent a sample cylinder 324. Sample cylinder 324 is similar to sample chamber 226 illustrated in FIG. 2. Sample cylinder 324 is where a urine sample collects and is allowed to settle for testing and analysis. Sample cylinder 324 may be transparent or have a section that is transparent such as a window running along the vertical axis of the cylinder. A transparent window may be located along a horizontal axis of cylinder 324 on top, bottom or both top and bottom of the cylinder. The window may be transparent to visible or non-visible light. Sample cylinder 324 may be made of at least a portion of glass or a transparent polymer such as polycarbonate. Sample cylinder 324 may be graduated to determine the volume of the urine sample deposited by a user. Sample cylinder 324 may also be referred to as a sample chamber, receptacle, container, cup, or other receiving device.

Sample cylinder 324 comprises an overflow 326. Overflow 326 may be in fluid communication with drain 308. Sample cylinder 324 further comprises one or more sensors 328. Multiple sensors can be placed along the cylinder 324 to detect information about the settled sections of urine. This includes the portion of urine where there is a larger concentration of particles near the bottom of the sample cylinder and the portion at the top with the clarified urine where there may be none or extremely low concentration of particles or solids in the urine. Alternatively, one or more sensors may be placed at a point to detect information as the urine exits the cylinder 324. Resistivity can be measured along length of the urine cylinder 324 equipped with resistivity probes to detect, for example, salinity, osmolality, and other information, such as pH, about the urine sample. Sensors 328 may comprise fiber optic sensors or one or more spectrometers. Sensors 328 may comprise one or more imaging sensors.

Analytical toilet 300 may further comprise an image capturing device, such as image capturing device 234 illustrated in FIG. 2, that is adjacent to cylinder 324. The image capturing device may further comprise a light source to illuminate the urine sample 330 and take both video and still images.

Toilet 300 further comprises a second valve 332. In a preferred embodiment, second valve 332 is a ball valve as depicted in FIG. 3. Valve 332 is located below cylinder 324 and may be used to drain the urine sample once testing and analysis has been completed. In a preferred embodiment, the inner diameter of sample cylinder 324 and inner diameter of valves 322 and 332 are approximately identical. The diameter may be larger than about 1 mm and may be in the range of about 1 mm to about 50 mm. In some instances, valve 332 may be located on a side of cylinder 324. Other valves that may be used for the first 322 or second valves 332 are solenoid valves, needle valves, butterfly valves, pinch valves, diaphragm valves, globe valves, angle body valves, or angle seat piston valves.

Valve 332 is adjacent to and in fluid communication with a cylinder drain passage 334. Cylinder drain passage 334 provides a pathway for urine to drain from cylinder 324 into drain 308 or the sewer. Overflow 326 can link with and drain into cylinder drain passage 334 as shown in FIG. 3. In some embodiments, toilet 300 may only comprise a single valve under cylinder 324 wherein valve 322 may not be necessary or required. Toilet 300 may further comprise a control system, such as control system 236 as previously described, to provide power to the sensors 328, image capturing devices, valves 322, 332, or other components.

Analytical toilet 300 may be operated to test and analyze a urine sample as follows. A user deposits a sample of urine into the urine collection area 310 in bowl 302. First valve 322, a ball valve is partially open to allow for urine to pass but not any solid materials. Sample cylinder 324 fills with the urine sample 330. As the urine sample collects in the cylinder 324, the sensors 328 detect the rate at which the cylinder is filled by the sample and takes a final volume reading once the fill rate reaches zero. Once the filling of the sample cylinder 324 is completed, the urine is allowed to settle over a period of time. Also, first valve 322 may also close during settling, testing and analysis to prevent possible contamination. The settling time may be in the range of about 0.5-60 min. The urine may be analyzed shortly after a user has excreted a sample of urine before any settling of the urine has occurred. In some instances, the urine may be analyzed after partial settling of the urine has been completed. In other instances, the settling time may be longer than 60 min in order to attain substantially complete settling of a urine sample. The sensors continue to monitor the urine 330 while an image capturing device illuminates the urine sample 330 and records the settling process. Once the pre-determined settling time is completed and the sensors collect the information, the second valve 332 is opened and allows the urine sample 330 to pass through passage 334 and be drained into the sewer. The urine sample can be drained intermittently or slowly to allow sensors located at the base of the sample cylinder 324 to take multiple readings of the various densities. Once the urine is drained, the urine collection area 310, valve 322, sample cylinder 324, second valve 332, and passage 334 (which are all in fluidic communication) are rinsed and/or sanitized with cleansing fluids.

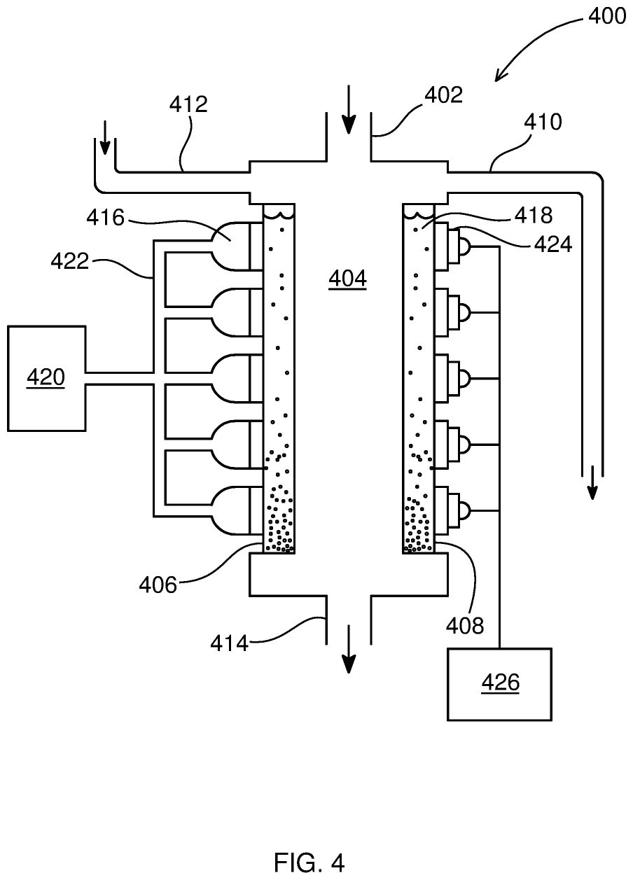

FIG. 4 illustrates a cross-sectional view of a urinalysis system 400 in an analytical toilet, according to an embodiment of the disclosure. Urinalysis system 400 comprises an inlet 402 from a urine collection area that is in fluidic communication with a urine collection chamber 404. Urine collection chamber 404 comprises a first transparent window 406 and a second transparent window 408 on opposing sides. Urine collection chamber 404 comprises a urine overflow 410 and an inlet 412 to dispense one or more reagents, buffers, wash fluids or other fluids from a dispenser into the collection chamber 404 from a reagent dispensing system. A one-way valve may be located at the location where inlet 412 enters chamber 404. A one-way valve would prevent urine from a high volume urination event by a user to enter inlet 412. Preferably, a high volume of urine would be drained out by overflow 410. Urine collection chamber 404 comprises a drain 414 to drain the urine sample, reagents, wash fluids and other fluids once analysis of a urine sample is completed.

Urine collection chamber 404 comprises one or more electromagnetic radiation emitters 416. The electromagnetic radiation emitters 416 emit electromagnetic radiation of pre-determined wavelength and frequency towards a urine sample 418 through one or more transparent windows 406, 408. One or more wavelengths or frequencies may be used for analysis of a urine sample, such as a continuum of wavelengths or frequencies. Five radiation emitters 416 are depicted in FIG. 4 for illustrative purposes only as one or more emitters may be used. The emitters are arranged along the height of collection chamber 404 along the vertical axis. In a preferred embodiment, the radiation emitters 416 may be arranged in an equidistant manner. Radiation emitters 416 emit electromagnetic radiation through a conduit 422 from a spectrometer or spectrophotometer 420. Fiber optic cables may be used as a conduit for electromagnetic radiation to be passed from a spectrometer or spectrophotometer 420 to the radiation emitters 416.

Opposing the emitters 416 on the opposite side of collection chamber 404 are one or more sensors 424. The sensors 418 receive the electromagnetic radiation that has passed through the urine sample 418 and through the second transparent window 408. FIG. 4 illustrates a preferred embodiment wherein one sensor opposes one emitter 416 and wherein the sensors 424 are arranged in an equidistant manner with spacing approximately equal to the emitters 416. The sensors 424 are further arranged along the height of chamber along the vertical axis. Urinalysis system 400 further comprises an analysis system 426 that collects and processes the electromagnetic radiation received by the sensors 424. System 426 may then transmit the data to a central processing unit (CPU) to process the data collected by the system 426.

By placing emitters 416 and sensors 418 along the vertical axis of the urine collection chamber 404, the analysis system 426 may be used to measure the temperature, sugar content and protein content of a urine sample at different sections. Sugars that may be detected and measured include glucose, sucrose or fructose. High glucose levels, for example, may be an indicator of glycosuria. In other embodiments, other sensors may be used and arranged accordingly to measure specific gravity, resistivity, pH, salinity, or osmolality at different sections of settled urine within the urine collection chamber 404. A combination of sensors may also be used to measure temperature, sugar content and protein content, blood, mucous, epithelial cells, fat, chyle, bacterial content, viral content, specific gravity, resistivity, pH, salinity, or osmolality at different sections of a urine sample in urine collection chamber 404. The sensors 418 may comprise one or more imaging sensors.

Analysis system 400 may be a refractometer-based system to project light into a urine sample in urine chamber 404 and determine the density of the urine at one or more sections. Analysis system 400 may be a colorimetric-based system to determine the color of the urine sample at one or more sections.

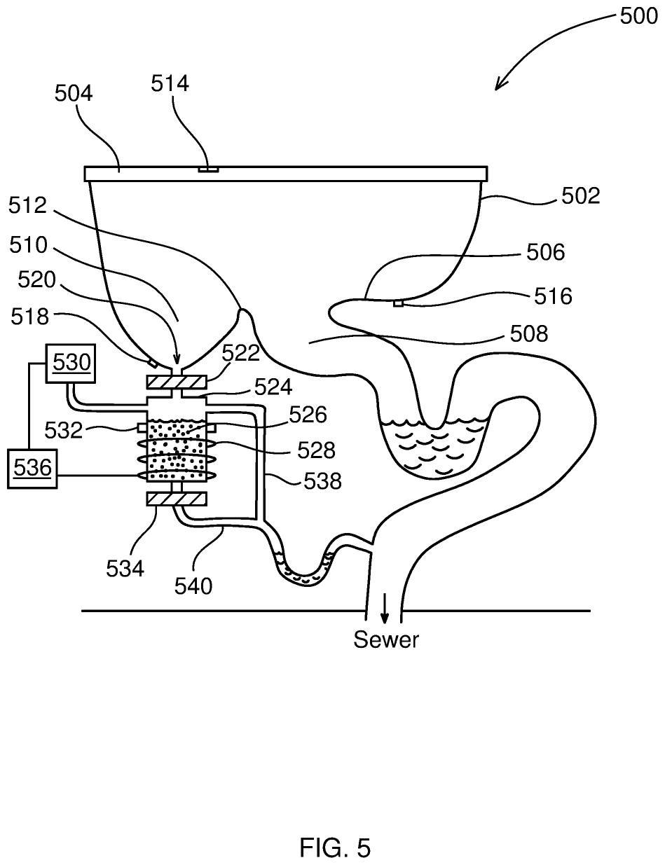

FIG. 5 illustrates a cross-sectional view of a urine analysis system with a heating element in an analytical toilet 500, according to an embodiment of the disclosure. Analytical toilet 500 comprises a bowl 502, seat 504, fecal shelf 506, drain 508, urine collection area 510, partition 512, one or more seat sensors 514, fecal shelf sensors 516, or urine collection area sensors 518. In some instances, there may be no seat sensors 514, fecal shelf sensors 516, or urine collection area sensors 518 in analytical toilet 500.

Toilet 500 illustrated in FIG. 5 further comprises a urine collection port 520 that is preferably located at the base of urine collection area 510. Port 520 may further comprise a device such as a filter or mesh screen to prevent solid material, such as feces or toilet paper, from passing through. An infra-red (IR) sensor may be adjacent to collection port 520 to determine the temperature of the urine sample to understand core body temperature of the user. Toilet 500 comprises a first valve 522 that is in fluid communication with bowl 502, area 510 and port 520. Valve 522 may be a solenoid valve, pneumatic valve, ball valve, or other form of valve. Toilet 500 comprises a urine sample container 524 such as a cylinder, chamber or other type of urine receptacle. Sample chamber 524 is similar to sample chamber 226 or cylinder 324 or chamber 404 as illustrated in FIGS. 2, 3, and 4 respectively. Sample chamber 524 is where a urine sample 526 collects and is allowed to settle for testing and analysis. Sample chamber 524 may be transparent or have a section that is transparent such as a window running along the vertical axis of the chamber. A transparent window may be located along a horizontal axis of chamber 524 on top, bottom, or both top and bottom of the chamber. Sample chamber 524 may be made of at least a portion of glass or a transparent polymer such as polycarbonate. Sample chamber 524 may be graduated to determine the volume of the urine sample deposited by a user. Sample chamber 524 may also be referred to as a sample chamber, receptacle, container, cylinder, or other receiving device.

Sample chamber 524 further includes a heating element 528 to heat urine sample 526. A heating device 528 may be configured to heat or even boil a urine sample to drive off water and concentrate the sample. Additionally, vapors driven out of the urine sample during heating may be analyzed, such as by a gas chromatography (GC), gas chromatography-mass spectrometry (GC-MS) device, or other gas sniffing device. Heating element 528 comprises heating coils as depicted in FIG. 5. Heating coils may be wrapped around at least a portion of sample chamber 524. Heating coils may be resistive coils. Heating element 528 may comprise an IR heater. Heating element 528 may comprise a hot stage located at the base of sample chamber 524. The heating element 528 may heat the urine sample at a rate of at least about 0.1° C./min. The heating element 528 may heat the urine sample at a rate in the range of about 0.1-20° C./min. The temperature range the heating element 528 may be used to heat a urine sample is about room temperature up to about 100° C. Analysis of the vapors driven may include detection of volatile organic compounds (VOCs) such as illicit or pharmaceutical drugs or biomarkers.

In some embodiments, heating element 528 may be used to heat a urine sample using a temperature ramp. As the temperature of the urine sample is increased, continuous measurement of properties of the urine may be taken. Different temperatures may also be held for pre-determined lengths of time to better evaluate a urine sample. For example, specific gravity, density, resistivity, pH, salinity, or osmolality, or a combination thereof, may be measured as a function of time and temperature. The precipitation or dissolution of one or more analytes, such as sugars, proteins, bacteria, viruses, blood, mucous, fat, chyle, leucocytes, epithelial cells, or other biomarkers, may also be detected as a function of temperature.

Analytical toilet 500 further comprises a reagent dispensing system 530 in fluidic communication with sample chamber 524. Reagent dispensing system 530 may be used to dispense one or more reagents into a urine sample before or after the sample is heated. The dispensing system may comprise a microfluidic, capillary, diaphragm, piston, screw, rotary, or peristaltic dispensing system. Reagent dispensing system 530 may comprise a one-way valve to prevent urine from chamber 524 to enter as a result of a high-volume urination event. Reagents may be used in addition to one or more analytical devices to aid identify one or more analytes, such as sugars, proteins, bacteria, viruses, blood, mucous, fat, chyle, leucocytes, epithelial cells, or other biomarkers that may be present in the urine. Analytical toilet and urinalysis embodiments 200 and 300 described previously herein may also comprise a reagent dispensing system and one or more analytical devices for testing and analysis of urine samples. Reagents may include buffers or solvents. The buffer may have a pH in the range of about 1-12. For example, for a pH range of about 1-2.2, HCl/KCl may be used as a buffer. For a pH range of about 2.2-3.6, glycine/HCl may be used as a buffer. For a pH range of about 2.2-4.0, potassium hydrogen phthalate/HCl may be used as a buffer. For a pH range of about 3.0-6.2, citric acid/sodium citrate may be used as a buffer. For a pH range of about 3.7-5.6, sodium acetate/acetic acid may be used as a buffer. For a pH range of about 4.1-5.9, potassium hydrogen phthalate/NaOH may be used as a buffer. For a pH range of about 5.5-6.7, 2-(N-morpholino) ethanesulfonic acid (MES) may be used as a buffer. For a pH range of about 5.8-7.2, bis-tris methane (BIS-TRIS) may be used as a buffer. For a pH range of about 5.8-8.0, phosphate buffer (PBS) may be used as a buffer. For a pH range of about 6.0-7.2, N-(2-acetamido) iminodiacetic acid (ADA) may be used as a buffer. For a pH range of about 6.1-7.5, piperazine-N,N′-bis(2-ethanesulfonic acid) (PIPES) or N-2-aminoethanesulfonic acid (ACES) may be used as a buffer. For a pH range of about 6.2-7.6, 3-morpholino-2-hydroxypropanesulfonic acid (MOPSO) may be used as a buffer. For a pH range of about 6.3-9.5, 1,3-bis(tris(hydroxymethyl)methylamino)propane (BTP) may be used as a buffer. For a pH range of about 6.4-7.8, BES may be used as a buffer. For a pH range of about 6.5-7.9, 3-(N-morpholino)propanesulfonic acid (MOPS) may be used as a buffer. For a pH range of about 6.8-8.2, 2-[(2-Hydroxy-1,1-bis(hydroxymethyl)ethyl)amino]ethanesulfonic acid (TES) or 4-(2-hydroxyethyl)-1-piperazineethanesulfonic acid (HEPES) may be used as a buffer. For a pH range of about 6.9-8.3, 4-(N-morpholino)butanesulfonic acid (MOBS) may be used as a buffer. For a pH range of about 7.0-8.2, 3-(N,N-Bis[2-hydroxyethyl]amino)-2-hydroxypropanesulfonic acid (DIPSO) or 2-Hydroxy-3-[tris(hydroxymethyl)methylamino]-1-propanesulfonic acid (TAPSO) may be used as a buffer. For a pH range of about 7.0-9.0, 2-amino-2-(hydroxymethyl)-1,3-propanediol (Trizma base) may be used as a buffer. For a pH range of about 7.1-8.5, 4-(2-Hydroxyethyl)piperazine-1-(2-hydroxypropanesulfonic acid) Hydrate hydrate (HEPPSO) may be used as a buffer. For a pH range of about 7.2-8.5, piperazine-1,4-bis(2-hydroxypropanesulfonic acid) dihydrate (POPSO) may be used as a buffer. For a pH range of about 7.4-8.8, tricine may be used as a buffer. For a pH range of about 7.5-8.9, diglycine (Gly-Gly) may be used as a buffer. For a pH range of about 7.6-9.0, 2-(bis(2-hydroxyethyl)amino)acetic acid (BICINE) or N-(2-hydroxyethyl)piperazine-N′-(4-butanesulfonic acid) (HEPBS) may be used as a buffer. For a pH range of about 7.7-9.1, [tris(hydroxymethyl)methylamino]propanesulfonic acid (TAPS) may be used as a buffer. For a pH range of about 7.8-9.7, 2-Amino-2-methyl-1,3-propanediol (AMPD) or N-tris(Hydroxymethyl)methyl-4-aminobutanesulfonic acid (TABS) may be used as a buffer. For a pH range of about 8.3-9.7, N-(1,1-Dimethyl-2-hydroxyethyl)-3-amino-2-hydroxypropanesulfonic acid (AMPSO) may be used as a buffer. For a pH range of about 8.6-10.0, N-cyclohexyl-2-aminoethanesulfonic acid (CHES) may be used as a buffer. For a pH range of about 8.6-10.3, N-cyclohexyl-2-hydroxyl-3-aminopropanesulfonic acid (CAPSO) may be used as a buffer. For a pH range of about 9.0-10.5, 2-Amino-2-methyl-1-propanol (AMP) may be used as a buffer. For a pH range of about 9.7-11.1, N-cyclohexyl-3-aminopropanesulfonic acid (CAPS) may be used as a buffer. For a pH range of about 10.0-11.4, 4-(cyclohexylamino)-1-butanesulfonic acid (CABS) may be used as a buffer.

Other reagents include sulfosalicylic acid for testing of proteinuria, CuSO₄, or benedict's reagent to test for sugars; sodium nitroprusside (SNP) to detect acetone or acetoacetic acid; or acetic acid to detect phosphates, carbonates, or proteins. The one or more reagents may cause a color change in the urine sample that can be detected by a refractometer, spectrophotometer or spectrometer. The color detected may be an indicator of a health condition of the kidneys, diabetes, or other diseases.

Analytical toilet 500 further comprises one or more sensors 532 adjacent sample chamber 524. Sensors 532 may be used to collect information on the sample such as resistivity, pH, salinity, or osmolality. Sensors 532 may comprise one or more imaging sensors. An image capturing device, such as image capturing device 234 or a spectrometric or spectrophotometric sensor system 400 as previously described herein, may also be located adjacent sample chamber 524 to collect information while the urine sample 526 settles over a pre-determined period of time. Video or still images may be taken of the settling urine. The image capturing device may also include an illumination system to illuminate the urine sample during settling and analysis. Sample chamber 524 may include a temperature sensor such as a thermometer or thermocouple. Light emitting diodes (LEDs) of various frequencies and wavelengths are emitted and illuminate the urine sample at various depths or sections in the sample chamber 524 to collect information on the sample.

Analytical toilet 500 further comprises a second valve 534. Valve 534 is used to drain the urine sample once analysis and testing has been completed. Valves that may be used for the first 522 or second valves 534 are ball valves, solenoid valves, needle valves, butterfly valves, pinch valves, diaphragm valves, globe valves, angle body valves, or angle seat piston valves. In some embodiments, analytical toilet may only comprise the second valve 534 but not the first valve 522. In this instance, urine may freely drain from urine collection area 510 through port 520 and into sample chamber 524.

Analytical toilet 500 further comprises a control system 536. Control system 536 provides power to one or more sensors 532, heating element 528, valves 522 or 534, an image capturing device, analytical devices, or other components used to test and analyze a urine sample located in chamber 524. System 536 may also be capable of providing processing and storage of the information collected by the sensors and analytical devices.

Sample chamber 524 comprises an overflow 538. Overflow 538 is used in the instance for a high volume urination event by a user. Overflow 538 can also be used as vent or pressure release if the urine sample is heated or boiled by heating device 528. Sample chamber 524 comprises a drain passage 540 wherein the urine sample may be drained to a sewer or other location once analysis is completed. The urine sample can be drained intermittently or slowly to allow the sensors at the base of the sample chamber 524 to take multiple readings of the various densities. Once the urine sample is drained, the urine collection area 510, port 520, valves 522 and 534, chamber 524, reagent dispensing unit 530, overflow 538, drain 508, and drain passage 540 are all in fluidic communication.

Analytical toilet 500 may be operated to test and analyze a urine sample as follows. A user deposits a sample of urine into the urine collection area 510 in bowl 502. First valve 522, is partially open to allow for urine to pass but not any solid materials. Sample chamber 524 fills with the urine sample 526. As the urine sample collects in chamber 524, the sensors 532 detect the rate at which the chamber is filled by the sample and takes a final volume reading once the fill rate reaches zero. Once the filling of the sample chamber 524 is completed, the urine is allowed to settle over a period of time. Also, first valve 522 may also close during settling, testing and analysis to prevent possible contamination. The settling time may be in the range of about 0.5-60 min. In some instances, the settling time may be longer than 60 min. The sensors continue to monitor the urine 526 while an image capturing device illuminates the urine sample 526 and records the settling process. The heating element 528 may then be activated to boil a portion of urine sample 526 while the vapors being driven off during the heating process are collected and analyzed by a GC-MS system. One or more reagents may be dispensed by system 530 into the urine sample. Vapors may be tested and could be indicators for cystitis (bladder inflammation), dehydration, diabetic ketoacidosis, gastrointestinal-bladder fistula, maple syrup urine disease, metabolic disorder, type 2 diabetes, or a urinary tract infection (UTI).

Once the pre-determined settling time, boiling time and reagents are dispensed is completed and the sensors collect information, the second valve 534 is opened and allows the urine sample 526 to pass through passage 540 and be drained into the sewer. The bowl 502, urine collection area 510, port 510, valve 522, sample chamber 524, second valve 534, and passage 540 (which are all in fluidic communication) are rinsed and/or sanitized with cleansing fluids in preparation for the next user.

In one embodiment, analytical toilet system 500 with a heating device may incorporate a spectrometric, spectrophotometric, refractometric, or colorimetric analysis system 400 illustrated in FIG. 4 and previously described herein.

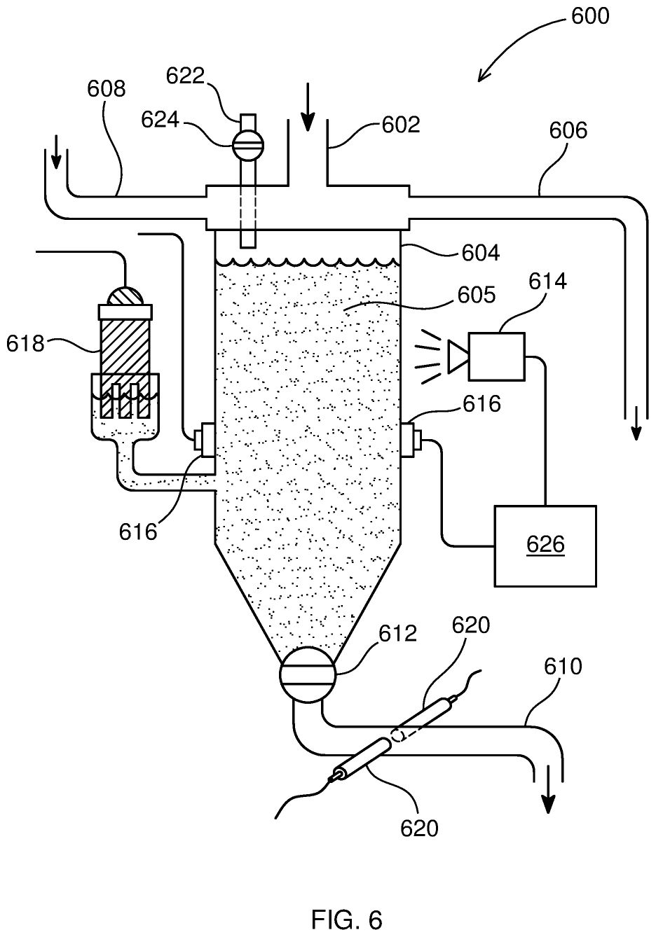

FIG. 6 illustrates a cross-sectional view of a urinalysis system 600 in an analytical toilet, according to an embodiment of the disclosure. Urinalysis system 600 comprises an inlet 602 from a urine collection area that is in fluidic communication with a urine collection chamber 604. Urine collection chamber 604 collects a sample of urine 605 for analysis. Urine collection chamber 604 is transparent to visible light or other electromagnetic radiation. The chamber may be made of glass, plastic or quartz. Urine collection chamber 604 comprises a urine overflow 606 and an inlet 608 to dispense one or more reagents, buffers, wash fluids or other fluids from a dispenser into the collection chamber 604 from a reagent dispensing system. A one-way valve may be located at the location where inlet 608 enters chamber 604. A one-way valve would prevent urine from a high-volume urination event by a user to enter inlet 608. Preferably, a high volume of urine would be drained out by overflow 606. Urine collection chamber 604 comprises a drain 610 to drain the urine sample, reagents, wash fluids and other fluids once analysis of a urine sample is completed. Drain 610 may be in fluidic communication with a sewer. Urinalysis system 600 further comprises a valve 612. Valve 612 may be closed during an excretion event by a user and during analysis of the urine. The valve 612 may be opened to drain the sample of urine once analysis is completed.

Urinalysis system 600 comprises an image capturing device 614 as previously described herein. Urinalysis system 600 comprises two or more electrodes 616. Electrodes 616 may be embedded in the chamber 604 to run a current through a urine sample. Electrodes 616 may be used to determine resistivity or conductivity. Not only could the electrodes 616 be used for analytical purposes, but by electrolyzing the urine, sodium ions in the urine may react and create a cleaning agent. This can be used to help sanitize the chamber 604 between uses. Electrodes 616 may be located anywhere in system 600.

Urinalysis system 600 comprises an ion detection device 618. Ion detection device 618 may be an ion selective electrode device or an ion chromatography device. Ion detection device 618 may be used to detect one or more ions found in urine in a healthy or non-healthy user. These ions may include Na⁺, K⁺, Ca²⁺, Mg²⁺, PO₄³⁺, Cl⁻, SO₄²⁻, HCO₃⁻, C₅H₃N₄O₃⁻ (urate), or NH₄⁺. The ion detection device can be periodically calibrated using a buffer solution.

Urinalysis system 600 comprises a variable light path spectroscopic analysis system 620. The spectroscopic analysis system 620. Comprises an electromagnetic radiation emitter that can emit light of various wavelengths and frequencies and an electromagnetic radiation sensor that detects the light that has passed through a urine sample to determine what light has been absorbed or scattered. The distance or gap between the emitter and sensor may be varied, such as in the range of about 1 mm to about 20 mm. Preferably the gap is in the range of about 1-3 mm. The gap may be optimized depending on the wavelength of electromagnetic radiation used. In urinalysis embodiment 600, the spectroscopic analysis system 620 is placed such that when urine is drained through drain 610, the urine may be analyzed. The light path between the radiation emitter and sensor is about the distance of the diameter of drain 610. This distance may be varied. Urinalysis system 600 may comprise one or more imaging sensors.

Urinalysis system 600 comprises a drawing port 622 for sample drawing. Port 622 comprises a valve 624 that may be kept closed except when to draw a sample. A needle and syringe or other device may be used to draw or extract a sample through the port 622. This allows for urine samples to be transported to other analysis modules or manually extracted for tests that cannot currently be done automatically in the toilet. If a test needs to be run outside the chamber 604, this eliminates the need for a user to excrete a sample of urine into a cup.

Urinalysis system 600 comprises a control system 626 to provide power to the components that require power for operation such as valve 612, image capturing device 614, electrodes 616, ion detection device 618, spectroscopic analysis system 620, valve 624, or other components. In a preferred embodiment, control system 626 is in electronic communication with a processor and a computer terminal or other electronic display.

An ion detection device 618, variable light path spectroscopic analysis system 620, electrodes 616, or port 622 and valve 624 may be combined with other analytical toilet and urinalysis systems described herein.

Any of analytical toilet systems 200, 300, 400, 500, or 600 described herein may be used to determine specific gravity of a urine sample. A urine specific gravity test compares the density of urine to the density of water. This test can help determine how well the kidneys of a user are diluting urine. Urine that is too concentrated could mean that the kidneys are not functioning properly or that the user is not drinking enough water. Urine that is not concentrated enough can mean that a user has a rare condition called diabetes insipidus, which causes thirst and the excretion of large amounts of diluted urine. Urine specific gravity results will fall between 1.002 and 1.030 if kidneys are functioning normally. Urine specific gravity results greater than 1.030 indicates extra substances in the urine such as sugars, protein, white or red blood cells, bilirubin, crystals, mucous, fat, chyle, leucocytes, epithelial cells, or bacteria which may indicate the kidneys are not functioning properly. In this instance, the user or a health care professional may be notified.

Any of analytical toilet systems 200, 300, 400, 500, or 600 described herein may comprise an image capturing device or a spectrometric or spectrophotometric sensor system that can detect the color, cloudiness, or clarity of the urine sample. For example, if cloudiness is detected in a urine sample this may be indicative of phosphorus, pyuria, chyluria, lipiduria, or hyperoxaluria in the user. A brown color may be indicative of bile pigments or myoglobin. A brownish-black color may be indicative of bile pigments, melanin, or methemoglobin. A green or blue color may be indicative of pseudomona UTI (urinary tract infection) or biliverdin. An orange color may be indicative of bile pigments. A red color may be indicative of hematuria, hemoglobinuria, myoglobinuria, or porphyria. A yellow color may be indicative of concentrated urine such as dehydration.

Any of analytical toilet systems 200, 300, 400, 500, or 600 described herein may comprise a device to insert a dipstick into the urine. Microscopic analyses of a urine sample may be carried out by a dipstick. The dipstick may be used to determine pH, protein content, glucose, ketones, blood, bilirubin, urobilinogen, nitrite, leukocytes, or other biomarkers. A dipstick may be from a cartridge of dipsticks loaded into the analytical toilet. The dipstick may be removed manually or automatically once a test of a user's urine is completed.

The dipstick may be used to determine the pH and may be capable of the double indicator method (methyl red and bromthymol blue) that covers the entire range of urine pH. The pH of urine is an indication of the kidney's ability to maintain a normal plasma pH.

The dipstick may be used to detect protein. The protein test is based on a change in color of a pH indicator (e.g. tetrabromophenol blue) in the presence of varying concentrations of protein when the pH is held constant. The reagent pad contains an indicator and a buffer that holds the pH of the pad at approximately 3. Yellow indicates undetectable protein. The color of positive reactions ranges from yellow-green to green to green-blue. Proteins may be detected in the range of about 5 mg/dL up to about 2000 mg/dL. Albumin and globulins may be detected.

The dipstick may be used to test for elevated levels of glucose and is based on a double enzyme method employing glucose oxidase and peroxidase. Color change ranges from green to brown.

The dipstick may be used to detect ketones. A nitroprusside reaction may be used to test for acetoacetic acid. The reaction of acetoacetic acid with nitroprusside results in the development of color ranging from buff pink to shades of purple. Color reactions are categorized as trace, small, moderate and large that correspond to ketone concentrations of 5, 15, 40 to 80 and 80 to 160 mg/dL of urine, respectively. Dipsticks reliably detect ketone concentrations of 40 mg/dL or more, so moderate and large readings do not need to be confirmed. Trace and small readings should be confirmed by using Acetest. The detection level for Acetest tablets is 20 mg/dL. The presence of ketonuria does not signal the need to do further microscopic evaluation.

The dipstick may be used to detect blood. The dipstick test for blood is based on the peroxidase-like activity of hemoglobin. Red cells are lysed on contact with the strip, allowing free hemoglobin to catalyze the liberation of oxygen from organic peroxide. Tetramethylbenzidine is oxidized, producing a color change from orange to green-blue. If intact red cells do not lyse, they may produce speckles on the pad. The sensitivity of dipsticks for hemoglobin is 0.015 to 0.062 mg/dL. This concentration corresponds to 5 to 21 RBCs/uL or 1 to 4 RBCs/hpf of concentrated urine sediment.

The dipstick may be used to detect bilirubin. The bilirubin dipstick test detects conjugated bilirubin and has a sensitivity of 0.5 to 1.0 mg/dL. This test is based on the binding of conjugated bilirubin to diazotized salts fixed in the test pad in a strong acidic environment to produce a colored compound that is various shades of tan or magenta. Positive dipstick tests are confirmed with the Ictotest. Normal adult urine contains about 0.02 mg/dL of bilirubin, which is not detectable by even the most sensitive methods. Confirmation of positive dipstick bilirubin results is most valuable when the urine specimen is pale yellow.

The dipstick may be used to detect urobilinogen. The dipstick may use para-dimethylaminobenzaldehyde in a strongly acid medium to test for urobilinogen. A positive reaction produces a pink-red color. Urobilinogen is normally present in urine at concentrations up to 1.0 mg/dL. A result of 2.0 mg/dL represents the transition from normal to abnormal. False positive results can be caused by medications such as para-aminosalicylic acid, antipyrine, chlorpromazine, phenazopyridine, phenothiazine, sulfadiazine, and sulfonamide. High nitrite concentrations can cause false negative reactions. Pigmented urine can interfere with detection of urobilinogen. Conjugated bilirubin is normally excreted into the bowel where bacteria metabolize it to urobilinogen. Urobilinogen is partially reabsorbed from the gut and excreted in the urine. A positive test indicates increased bilirubin delivery to the gut. Hepatitis produces positive urine bilirubin and urobilinogen. Biliary tract obstruction results in positive urine bilirubin but negative urobilinogen. Hemolytic anemia causes negative urine bilirubin and positive urobilinogen. Bilirubin and urobilinogen tests are valuable in detecting hemolysis, hepatic dysfunction, and biliary obstruction. The results of these two tests should be interpreted together. Bilirubin is unstable and rapidly decomposes during exposure to light. False negative reactions are common if urine is not tested shortly after collection. Chlorpromazine (Thorazine) and selenium can produce false negative results.

The dipstick may be used to detect leukocytes in a urine sample. Pyuria (the presence of leukocytes in the urine) can be detected using the leukocyte esterase reagent strip test. The assay is based on the chemical detection of esterases, which are enzymes contained within the azurophilic granules of polymorphonuclear leukocytes. Esterase level is directly proportional to the number of leukocytes present in a urine sample. The basis of the chemical reaction is the hydrolysis of an ester to form an aromatic alcohol and acid. The aromatic compound combines with a diazonium salt to form an azo-dye that changes to purple. Color intensity read at two minutes is proportional to the number of granulocytes in a sample.

The dipstick may be used to detect nitrite in a urine sample. The nitrite test is a rapid, indirect method for detecting bacteriuria. The reaction principle is based on bacterial reduction of dietary nitrate, which is normally present in urine, to nitrite, which is not normally present. Nitrite reacts with para-arsanilic acid on the dipstick to form a diazonium compound that reacts with a benoquinoline to form a pink color.

Any of analytical toilet systems 200, 300, 400, 500 or 600 described herein may further comprise a trigger sensor. A trigger sensor initiates the analysis of a urine sample once a urination event by a user is sensed. The urination event may be sensed by measuring the temperature of the urine by a temperature sensor, movement of urine by a motion sensor, increase in volume of a urine sample by movement of a float in the sample chamber in a vertical direction, or a pause in the movement of a float which indicates no more urine is being excreted by a user. The pause may be about a 5 sec or more pause. A trigger sensor may aid in operating the analytical toilet in a more automated way. This would reduce or eliminate the possibility of human error as a factor in the testing and analysis. An excretion profile, or more specifically a urination profile, of the user may be generated from the data collected during the urination event.

Any of the analytical toilet embodiments, 200, 300, 400, 500 or 600 described herein may further comprise a device to measure the mass of a urine sample excreted by a user. Any of the urine sample collection chamber embodiments 226, 324, 404, 524 or 604 described herein may further comprise a balance or an integrated load cell. The load cell may be a hydraulic load cell, pneumatic load cell, strain-gauge load cell, canister load cell, bending beam load cell, helical load cell, fiber optic load cell, piezo-resistive load cell, shear beam load cell, ring load cell, pancake load cell, inductive load cell, reluctance load cell, or a magnetorestrictive load cell. The load cell may be located at the base of a urine sample collection chamber. The mass of the urine sample may be taken in combination with the volume of the sample as determined by a fluid level sensor to calculate the density.

Other devices may be integrated into the analytical toilet embodiments, 200, 300, 400, 500 or 600 described herein to measure density. For example, a vibrating tube densitometer, such as an oscillating U-tube, may be integrated. The oscillating U-tube is a technique to determine the density of liquids and gases based on an electronic measurement of the frequency of oscillation, from which the density value can be calculated. A density meter may be integrated to measure the density of a urine sample, such as a density meter manufactured by Anton Paar (Graz, Austria).

Any of analytical toilet systems 200, 300, 400, 500 or 600 described herein may be capable of completing a 24 hour urine test. By logging into an analytical toilet each time a user excretes urine over a 24 hour period, the data from multiple urine excretion events can be tracked and logged. The data may be compiled into a report by a data processor located within the analytical toilet. A “stone risk profile” and other information may be determined from the 24 hour test.

The toilet disclosed herein has many possible uses, including private and public use. Whether for use by one individual, a small group of known users, or general public use, the toilet can detect, monitor, and create one-time and/or trend data from analysis of urine. This data can be used to prompt a user to seek additional medical, health, wellness advice, or treatment; track or monitor a user or population's known condition; and provide early detection or anticipation of a contagious disease, injury or another condition of which a user or population may wish to be aware. The data may be sent directly to the health care professional of the user.

### EXAMPLE

### Example 1. Settled Urine Analysis in an Analytical Toilet

Example 1 is illustrative of a preferred method of urine analysis of a settled urine sample in an analytical toilet. The method comprises:
1. A user releasing a sample of excreta into an analytical toilet.
2. Urine is collected in the urine collection area.
3. Urine passes through the port and a first valve into the urine sample chamber.
4. First valve closes after the urine is collected and no more urine is received for a period of 15 sec.
5. An image capturing device illuminates the urine sample then the analytical toilet measures urine mass, volume, density, temperature, and color.
6. The urine is gravimetrically settled for a period of 30 min. The image capturing device records the settling event.
7. Sensors measure resistivity, salinity, pH and osmolality at different depths and sections of the sample at intervals of 1 min during the 30 min settling time.
8. Once the settling time is completed, the urine is examined visually for the presence of settled solids by images taken with the image capturing device and sent to the user.
9. The sample of urine is heated with a resistive heating element until a temperature of 90° C. is reached at a rate of 10° C./min.
10. Vapors emitted by the heated urine are tested using a GC-MS.
11. The bottom valve is opened, and the urine sample drains to the sewer. Washing and sterilization fluids are added to the bowl and urine collection area. The valves and sample chamber are cleaned and sterilized and prepared for the next user.

### Example 2. Urine Analysis in an Analytical Toilet

Example 2 is illustrative of a preferred method of urine analysis in an analytical toilet. The method comprises:
1) A user releasing a sample of excreta into an analytical toilet.
2) Urine is collected in the urine collection area.
3) Urine passes through the port and a first valve into the urine sample chamber.
4) First valve closes after the urine is collected and no more urine is received for a period of 15 sec.
5) An image capturing device illuminates the urine sample 1 sec after the first valve closes. Then the analytical toilet measures urine mass, volume, density, temperature, and color.
6) Sensors measure resistivity, salinity, pH and osmolality at different depths and sections of the sample.
7) Ion selective electrode system measures Na⁺, K⁺, Ca²⁺, Mg²⁺, PO₄³⁺, Cl⁻, SO₄²⁻, C₂H₃O₂⁻, HCO₃⁻, C₅H₃N₄O₃⁻, and NH₄⁺ content.
8) The bottom valve is opened, and the urine sample drains to the sewer. As the urine sample drains, it passes through a gap of 2 mm where a variable light path spectroscopic analysis system analyzes the urine sample for protein, virus, bacteria, and sugar content.
9) Once all the urine sample passes through the variable light path spectroscopic analysis system, washing and sterilization fluids are added to the bowl and urine collection area. The valves and sample chamber are cleaned and sterilized and prepared for the next user.

The invention has been described with reference to various specific and preferred embodiments and techniques. Nevertheless, it is understood that many variations and modifications may be made while remaining within the spirit and scope of the invention.

What is claimed is:

1. An analytical toilet comprising:
a bowl for receiving excreta from a user;
a urine collection chamber in fluid communication with the bowl;
a sensor for detecting properties of the urine after at least partial settling of the urine;
a valve adapted to release the captured urine from the urine collection chamber; and
a source of flush water to clean the urine collection chamber.

2. The analytical toilet of claim 1, wherein at least a portion of the urine collection chamber is transparent.

3. The analytical toilet of claim 1, wherein the sensor further comprises one or more sensors along the height of the urine collection chamber are adapted to detect properties of different sections of the at least partially settled urine.

4. The analytical toilet of claim 3, wherein the one or more sensors measures protein, viral, bacterial, illicit drug, pharmaceutical drug, blood, mucous, fat, chyle, leucocyte, epithelial cell, or sugar content in the urine.

5. The analytical toilet of claim 3, wherein the one or more sensors measures the specific gravity, resistivity, pH, density, salinity, or osmolality at different sections of the at least partially settled urine within the urine collection chamber.

6. The analytical toilet of claim 3, wherein the one or more sensors spectroscopically measures the properties of different sections of the at least partially settled urine.

7. The analytical toilet of claim 2, further comprising an image capturing device and is positioned to capture images through the transparent portion of the urine collection chamber.

8. The analytical toilet of claim 7, wherein the image capturing device records the settling of the urine sample in the urine collection chamber over a pre-determined time period.

9. The analytical toilet of claim 7, wherein the image capturing device is configured to capture images along the vertical axis of the at least partially settled urine.

10. The analytical toilet of claim 7, further comprising additional image capturing devices, wherein the image capturing device and the additional image capturing devices are positioned at different heights along the urine collection chamber to record different sections of the at least partially settled urine.

11. The analytical toilet of claim 1, wherein the urine collection chamber further comprises a fluid level sensor device.

12. The analytical toilet of claim 11, wherein the fluid level sensor is used to measure the volume of urine added to the urine collection chamber and the rate at which urine is added to the sample collection chamber during a urination event by a user to thereby determine a urination profile.

13. The analytical toilet of claim 1, further comprising an ion detection device, variable light path spectroscopic analysis system, electrodes, or a drawing port.

14. The analytical toilet of claim 1, further comprising a filter to prevent feces from entering the urine collection chamber.

15. The analytical toilet of claim 1, further comprising an overflow path for urine exceeding the volume of the urine collection chamber.

16. The analytical toilet of claim 1, further comprising a heating element to drive off water and concentrate the urine sample.

17. The analytical toilet of claim 16, wherein the heating element is a resistive coil, IR heater, or a hot stage.

18. The analytical toilet of claim 1, further comprising a reagent dispensing device to dispense one or more reagents into the urine collection chamber.

19. The analytical toilet of claim 18, wherein the reagent dispensing device is a microfluidic, capillary, diaphragm, piston, screw, rotary, or peristaltic dispensing device.

20. The analytical toilet of claim 18, wherein the one or more reagents comprises a buffer solution, sulfosalicylic acid, CuSO₄, Benedict's reagent, sodium nitroprusside, or acetic acid.
